(19) United States
(12) Patent Application Publication
(10) Pub. No.: US 2014/0128677 A1
(43) Pub. Date: May 8, 2014

(54) Enema and Coloscopy Aide

(71) Applicants: Janet Lenart, Fort Francis (CA); Joyce Pennarun, Fort Frances (CA)

(72) Inventors: Janet Lenart, Fort Francis (CA); Joyce Pennarun, Fort Frances (CA)

(21) Appl. No.: 13/954,231

(22) Filed: Jul. 30, 2013

Related U.S. Application Data
(60) Provisional application No. 61/677,110, filed on Jul. 30, 2012.

(51) Int. Cl. A61B 1/00; A61B 1/31; A61M 3/02

(52) U.S. Cl. CPC A61B 1/00154; A61M 3/0279; A61B 1/31
USPC 600/114; 604/514

(57) ABSTRACT

A colonoscopy or enema aide is described which decreases friction or “rub” on the anus. Specifically, the colonoscope or enema tube is placed through the device which is then inserted into the rectum. The device prevents air from escaping. The device comprises a circular base and a conical section extending from the base to a tip through which the scope or tube is passed during use.

### PRIOR APPLICATION INFORMATION

The instant application claims the benefit of U.S. Provisional Patent Application 61/677,110, filed Jul. 30, 2012.

### BACKGROUND OF THE INVENTION

When giving an enema, the patient often cannot retain the fluid that allows the enema to work properly. As will be appreciated by one of skill in the art, any fluid that escapes prevents the enema from working to its maximum potential.

Fluid may escape for a variety of reasons, for example but by no means limited to hemorrhoids, relaxed anuses, or simply from patient pushing out the fluid rather than holding due to an inability to understand the instructions (for example, young children or Alzheimer patients).

In some cases, patients are uncomfortable during the procedure and may require multiple enemas to achieve the same results that could have been attained with one “proper” enema.

The same holds true for other similar procedures requiring tube insertion, for example, for the colonoscope and the flexible sigmoid scope.

What is needed is a device that would assist a patient in retaining the necessary fluids and improve patient comfort so that these procedures could be more effectively carried out.

U.S. Pat. No. 5,578,017 teaches a device that includes an adhesive sheet which sticks to the anus. It is noted however that the anus is moist and there is the risk that an adhesive to the anus could remove skin on removal, causing bleeding.

U.S. Pat. No. 3,990,448 teaches a device which has a cone that sits right inside the rectum, in such a way that it can be moved. Consequently, it is possible that this device could be inadvertently pushed higher up, thereby injuring the bowel as a result.

### SUMMARY OF THE INVENTION

According to a first aspect of the invention, there is provided a combination comprising: a colonoscopy or enema aide comprising: an open base arranged to be fitted against the outside of an anal opening; and a conical section extending from the base, said conical section tapering to a tip, said conical portion arranged to be inserted into an anal opening; and a rectal scope or tube, said tip forming an opening for passing a scope or tube therethough.

According to another aspect of the invention, there is provided a method of administering a rectal tube to an individual in need of such treatment comprising: providing an aide comprising: an open base arranged to be fitted against the outside of an anal opening; and a conical section extending from the base, said conical section tapering to a tip, said conical portion arranged to be inserted into an anal opening; inserting a first end of the rectal tube through the tip of the conical portion of the aide such that the tip of the aide is proximal to the first end of the tube and the base is distal to the first end of the tube, said tube being in communication at a second end thereof with a reservoir of fluid; applying the aide to the individual such that the conical section of the aide is inserted into the anal opening of the individual and the base of the aide is fitted against the outside of the anal opening of the individual and the first end of the rectal tube is inserted into the colon at a proper depth; and administering the fluid to the individual.

The rectal tube may be an enema tube or a medical scope.

The fluid may be an enema fluid or air.

According to another aspect of the invention, there is provided a method of administering an enema to an individual in need of such treatment comprising: providing an enema aide comprising: an open base arranged to be fitted against the outside of an anal opening; and a conical section extending from the base, said conical section tapering to a bulbous tip, said bulbous tip forming an opening , said conical portion arranged to be inserted into an anal opening; inserting a first end of an enema tube through the opening of the conical portion of the enema aide such that the open tip of the enema aide is proximal to the first end and the base is distal to the first end, said enema tube being connected at a second end thereof to a reservoir of enema fluid; applying the enema aide to the individual such that the conical portion of the enema aide is inserted into the anal opening of the individual and the base of the enema aide is fitted against the outside of the anal opening of the individual and the first end of the tube is inserted into the colon at a proper depth; and administering the enema fluid to the individual.

It is of note that in some embodiments, the enema tube may be removed once the enema aide has been properly applied.

The enema is then administered for the prescribed period of time, subsequent to which the enema aide is removed and the enema fluid is voided by the patient.

According to a further aspect of the invention, there is provided a method of performing a colonoscopy on an individual in need of such treatment comprising:

providing a colonoscopy aide comprising: an open base arranged to be fitted against the outside of an anal opening; and a conical section extending from the base, said conical section tapering to a bulbous tip, said bulbous tip being open, said conical portion arranged to be inserted into an anal opening; inserting an insertion end of a colonoscope through the open tip of the conical portion of the colonoscopy aide such that the open tip of the colonoscopy aide is proximal to the insertion end and the base is distal to the insertion end; applying the colonoscopy aide to the individual such that the conical portion of the colonoscopy aide is inserted into the anal opening of the individual and the base of the colonoscopy aide is fitted against the outside of the anal opening of the individual and the insertion end of the colonoscope is inserted into the colon at an initial depth; and continuing the colonoscopy procedure.

In some embodiments, the bulbous tip may be open or the bulbous tip may be closed, that is, the bulbous tip may have a removable section that is removed for opening the bulbous tip for passing a scope or tube therethrough.

In some embodiments, the bulbous tip includes indicia or markings for cutting off a portion of the bulbous tip such that an opening of the desired size, for example, to accommodate an enema tube or a colonoscope, can be created.

### BRIEF DESCRIPTION OF THE DRAWINGS

FIG. 1 is a side view of the device with a colonoscope inserted therethrough.

FIG. 2 is a schematic diagram showing the combination in use.

FIG. 3 is a side view of an embodiment of the device arranged to be used on an already-inserted tube or colonoscope.

FIG. 4 is a bottom view of an embodiment of the device arranged to be used on an already-inserted tube or colonscope.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a device which assists a patient undergoing an enema or similar procedure in retaining the enema fluid throughout the procedure. The device also improves patient comfort throughout the procedure, as discussed below.

Specifically, the device is suited for individuals in need of an enema or undergoing a rectal procedure such as a colonoscopy or the like, particularly for such individuals who suffer from hemorrhoids, a relaxed anus or are otherwise incapable of retaining the fluid during the procedure.

Specifically, anuses relax due to side-effects of administered pharmaceuticals and/or other health issues such as hemorrhoids and fissures. This can allow fluid (air) to escape during an anal procedure which can interfere with bowel insufflation.

Accordingly, in one embodiment of the invention, “an individual in need of such treatment” as used in the context of the invention refers to an individual who has a relaxed anus or is otherwise incapable of retaining fluid during an anal procedure, as discussed herein.

Described herein is a device which decreases friction or “rub” on the anus. Specifically, the colonoscopy scope or enema tube is placed through the device which is then inserted into the rectum. The device prevents air from escaping, as discussed herein.

In one embodiment of the invention, there is provided a colonoscopy or enema aide comprising: an open base arranged to be fitted against the outside of an anal opening; and a conical section extending from the base, said conical section tapering to a tip, said conical portion arranged to be inserted into an anal opening, said tip arranged for passing a rectal scope or tube therethrough.

According to another aspect of the invention, there is provided the use of the above described colonoscopy or enema aide for carrying out a rectal procedure in a patient.

According to another aspect of the invention, there is provided the use of the above described enema aide for administering an enema to a patient.

According to another aspect of the invention, there is provided the use of the above described colonoscopy aide for carrying out a colonoscopy procedure on a patient.

According to another aspect of the invention, there is provided a method of administering a rectal tube to an individual in need of such treatment comprising: providing an aide comprising: an open base arranged to be fitted against the outside of an anal opening; and a conical section extending from the base, said conical section tapering to a tip, said conical portion arranged to be inserted into an anal opening; inserting a first end of the rectal tube through the tip of the conical portion of the aide such that the tip of the aide is proximal to the first end of the tube and the base is distal to the first end of the tube, said tube being in communication at a second end thereof with a reservoir of fluid; applying the aide to the individual such that the conical section of the aide is inserted into the anal opening of the individual and the base of the aide is fitted against the outside of the anal opening of the individual and the first end of the rectal tube is inserted into the colon at a proper depth; and administering the fluid to the individual.

The rectal tube may be an enema tube or a medical scope.

The fluid may be an enema fluid or air.

In another embodiment of the invention, there is provided a combination comprising: a colonoscopy or enema aide comprising: an open base arranged to be fitted against the outside of an anal opening; and a conical section extending from the base, said conical section tapering to a tip, said conical portion arranged to be inserted into an anal opening; and a rectal scope or tube.

In some embodiments, the rectal scope, for example, a colonoscope or other endoscope, or rectal tube, for example an enema tube or other such tube, is inserted into the aide, as discussed herein. As discussed below, the tip may initially be closed but arranged such that the tip can be opened for forming an opening or the tip may be open for passing the tube or scope therethrough, as discussed herein. Furthermore, in either case, the tip may include markings or indicia for facilitating the cutting of the tip such that an opening of the desired diameter can be quickly and easily generated. As discussed below, indicia or markings may be included for generating an opening suitable for accommodating an enema tube or a scope. As will be appreciated by one of skill in the art, a lubricant may be applied to the tube or scope prior to insertion into the opening which will improve movement of the tube relative to the device and will also provide some sealing function.

The base may be composed of plastic for example a somewhat rigid plastic and the base may have a substantially circular shape, although other suitable shapes may be used as well.

Preferably, the conical section includes markings for cutting the conical portion proximal to the tip to create or increase the diameter of the opening, as discussed herein. For example, the conical section may taper to a tip, for example a bulbous tip or other suitably shaped tip and that tip may be closed or open. However, the conical section may include graduated markings for cutting the conical section so as to create an opening in the conical section of a desired diameter. For example, the desired diameter may be slightly larger than an enema tube or slightly larger than a colonoscopy scope, as discussed above. As will be appreciated by one of skill in the art, this opening is at the tip of the conical section.

According to another aspect of the invention, there is provided a method of administering an enema to an individual in need of such treatment comprising: providing an enema aide comprising: an open base arranged to be fitted against the outside of an anal opening; and a conical section extending from the base, said conical section tapering to a tip, said conical portion arranged to be inserted into an anal opening; inserting a first end of an enema tube through the opening of the conical portion of the enema aide such that the tip of the enema aide is proximal to the first end and the base is distal to the first end, said enema tube being in communication at a second end thereof with a reservoir of enema fluid; applying the enema aide to the individual such that the conical section of the enema aide is inserted into the anal opening of the individual and the base of the enema aide is fitted against the outside of the anal opening of the individual and the first end of the tube is inserted through the tip of the conical section into the colon at a proper depth; and administering the enema fluid to the individual.

Once the enema fluid has been administered, the enema tube may be removed. Once the prescribed time period has passed, the enema aide is removed and the enema fluid is voided by the patient.

According to another aspect of the invention, there is provided a method of performing a colonoscopy on an individual in need of such treatment comprising: providing a colonoscopy aide comprising: an open base arranged to be fitted against the outside of an anal opening; and a conical section extending from the base, said conical section tapering to a tip, said conical portion ending in an open tip, said conical portion arranged to be inserted into an anal opening; inserting an insertion end of a colonoscope through the open tip of the conical portion of the colonoscopy aide such that the open tip of the colonoscopy aide is proximal to the insertion end and the base is distal to the insertion end; applying the colonoscopy aide to the individual such that the open tip of the colonoscopy aide is inserted into the anal opening of the individual and the base of the colonoscopy aide is fitted against the outside of the anal opening of the individual and the insertion end of the colonoscope is inserted into the colon at an initial depth; and continuing the colonoscopy procedure.

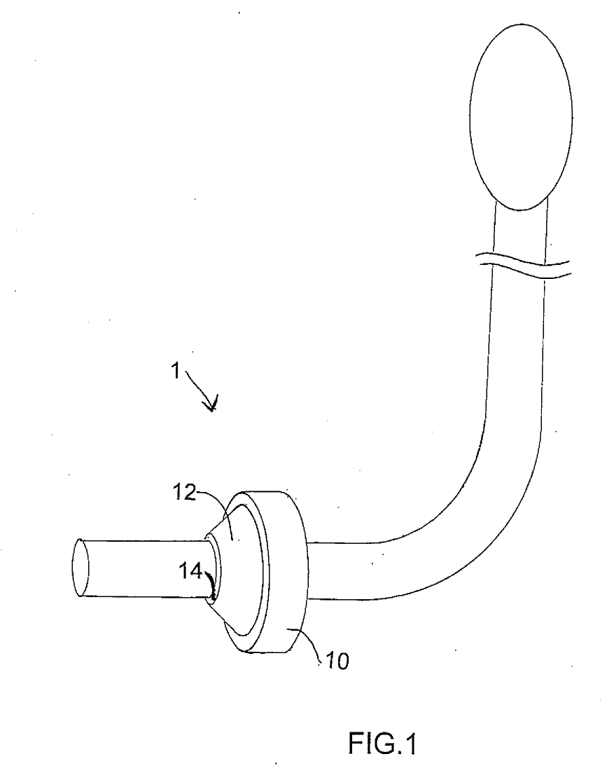

In a preferred embodiment, the device has the general structure as shown in FIG. 1. Specifically, the device 1 comprises a circular base 10 and a conical section 12 extending, that is, tapering or narrowing from the base to a flattened point 14. The flattened point may be open as shown in FIG. 1.

In a preferred embodiment, the conical section includes indicia along a side thereof for cutting or trimming off a portion of the conical section such that the flattened tip is removed and the tip or top portion of the conical section is opened to the desired diameter. That is, the flattened tip may be open or closed. The conical portion may include indicia printed thereon where the conical portion narrows to the flattened tip for cutting the flattened tip or conical section so that an opening of the diameter desired is generated at the tip of the device.

Preferably, the device or at least the conical section thereof is made of latex-free plastic.

In a preferred embodiment, the device has a structure similar to that of the nipple of a baby bottle.

In a preferred embodiment, the conical section includes markings along a side thereof for cutting the conical region so as to create an opening of for example 0.5 cm or 1.3 cm as well as suitable marking therebetween for creating an opening of a suitable diameter for insertion of a tube or scope, as discussed herein.

For use, the tip of the conical section may be cut so that an opening of the desired size is created. The device is then fitted over the scope or tube such that the base is distal to the open end of the tube relative to the open tip. In some embodiments, the tube or scope may be lubricated prior to being inserted through the opening in the conical section.

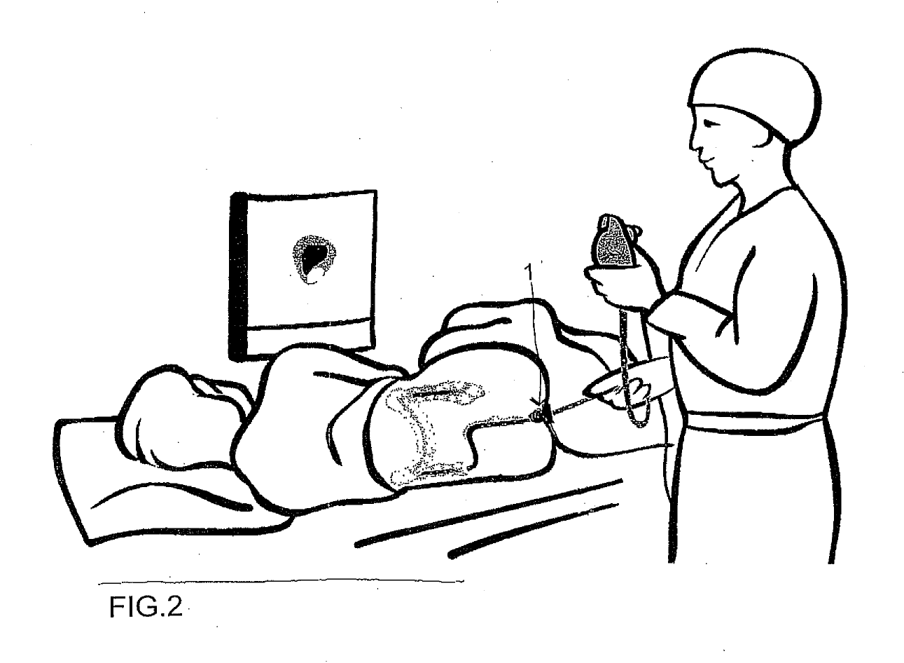

In use, the tube or scope is fed up from the plastic base through the opening in the conical section or nipple into the patient's anal opening and is held in place by the plastic base, as shown in FIG. 2. The nipple slides up into the rectal opening while the plastic base sits snuggly against the outside of the anus opening, assisting in preventing air or fluid from escaping the anus.

For a colonoscope or when using a flexible sigmoid scope, the doctor may now continue with a scope procedure that previously may have sent the patient home and they would have been required to come back for another procedure (ie Barium Enema), thus requiring another bowel prep. It allows the fluid (air) for insufflation to be kept in the rectum instead of released in the form of flatus, which allows for a better visualization of the bowel and allows the user of the scope to advance with a good visual.

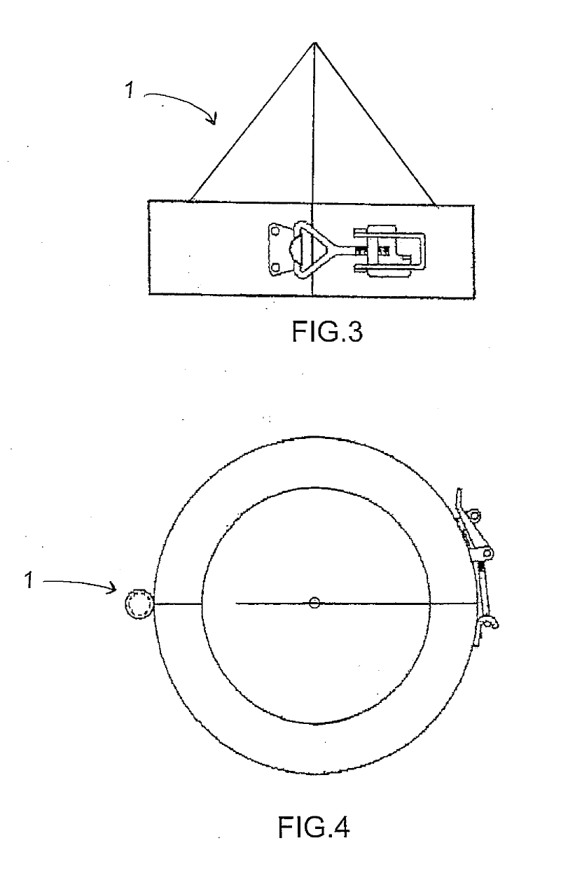

In some embodiments, the device is arranged such that it can be fitted over a tube or scope rather than fitted on one end of a tube or scope. As will be appreciated by one of skill in the art, in these embodiments, shown in FIGS. 3 and 4, the device is arranged to be used after a procedure has already started. For example, the plastic base may include a close-able opening, for example, a hinge, thereby providing an opening in the base for fitting the base around the tube or scope and the conical section may include an overlap portion. This overlap portion may include a closing means, for example, an adhesive strip or clasp for sealing the conical portion. In this arrangement, the device can be used if during a procedure it becomes clear that it is needed, thereby improving patient comfort and saving time for the health care professionals. Specifically, if it becomes clear during a rectal procedure that the device is needed, rather than removing the tube or scope, the hinge on the base can be opened and the base can be fitted over the tube or scope. The hinge is then closed. Next the overlap portion is fitted over the tube or scope and the closing means, for example, the clasp or adhesive strip is applied. The device is then fitted onto the patient as discussed above.

Preferably, the base is between 2 cm to 6 cm in diameter. In preferred embodiments, the plastic base is approximately 4.5 cm. As will be appreciated by one of skill in the art, the base may have to be larger, for example about 6 cm, in the case of an adult who has megacolon, or uses enemas a lot. Furthermore, as will be appreciated by one of skill in the art, smaller diameters are better suited for children.

This aide would save the health care system dollars as well as patient time, and comfort, as discussed above.

While the preferred embodiments of the invention have been described above, it will be recognized and understood that various modifications may be made therein, and the appended claims are intended to cover all such modifications which may fall within the spirit and scope of the invention.

1. A method of administering a rectal tube to an individual in need of such treatment comprising:
providing an aide comprising:
an open base arranged to be fitted against the outside of an anal opening; and
a conical section extending from the base, said conical section tapering to a tip, said conical portion arranged to be inserted into an anal opening;
inserting a first end of the rectal tube through the tip of the conical portion of the aide such that the tip of the aide is proximal to the first end of the tube and the base is distal to the first end of the tube, said tube being in communication at a second end thereof with a reservoir of fluid;
applying the aide to the individual such that the conical section of the aide is inserted into the anal opening of the individual and the base of the aide is fitted against the outside of the anal opening of the individual and the first end of the rectal tube is inserted into the colon at a proper depth; and
administering the fluid to the individual.

2. The method according to claim 1 wherein the rectal tube is an enema tube.

3. The method according 1 wherein the rectal tube is a medical scope.

4. The method according to claim 1 wherein the fluid is an enema fluid.

5. The method according to claim 1 wherein the fluid is air.

6. A method of administering an enema to an individual in need of such treatment comprising:
providing an enema aide comprising:
an open base arranged to be fitted against the outside of an anal opening; and
a conical section extending from the base, said conical section tapering to a tip, said conical portion arranged to be inserted into an anal opening;
inserting a first end of an enema tube through the tip of the conical portion of the enema aide such that the tip of the enema aide is proximal to the first end of the enema tube and the base is distal to the first end of the enema tube, said enema tube being in communication at a second end thereof with a reservoir of enema fluid;
applying the enema aide to the individual such that the conical section of the enema aide is inserted into the anal opening of the individual and the base of the enema aide is fitted against the outside of the anal opening of the individual and the first end of the tube is inserted into the colon at a proper depth; and
administering the enema fluid to the individual.

7. A method of performing a colonoscopy on an individual in need of such treatment comprising:
providing a colonoscopy aide comprising:
an open base arranged to be fitted against the outside of an anal opening; and
a conical section extending from the base, said conical section tapering to a tip, said conical portion arranged to be inserted into an anal opening;
inserting an insertion end of a colonscopy scope through the tip of the conical portion of the colonoscopy aide such that the bulbous tip of the colonoscopy aide is proximal to the insertion end and the base is distal to the insertion end;
applying the colonoscopy aide to the individual such that the conical section of the colonoscopy aide is inserted into the anal opening of the individual and the base of the colonoscopy aide is fitted against the outside of the anal opening of the individual and the insertion end of the colonoscopy scope is inserted into the colon at an initial depth; and
carrying out the colonoscopy procedure.
